(19) United States
(12) Patent Application Publication
(10) Pub. No.: US 2009/0226548 A1
(43) Pub. Date: Sep. 10, 2009

(54) METHOD OF PREVENTING, CONTROLLING AND AMELIORATING URINARY TRACT INFECTIONS USING A SYNERGISTIC CRANBERRY DERIVATIVE AND D-MANNOSE COMPOSITION

(75) Inventors: John A. Minatelli, Sanford, FL (US); W. Stephen Hill, Ocala, FL (US)

Correspondence Address: ALLEN, DYER, DOPPELT, MILBRATH & GILCHRIST P.A., 1401 CITRUS CENTER 255 SOUTH ORANGE AVENUE, P.O. BOX 3791, ORLANDO, FL, 32802-3791, US

(73) Assignee: U.S. Nutraceuticals, LLC d/b/a Valensa International, Eustis, FL (US)

(21) Appl. No.: 12/426,500

(22) Filed: Apr. 20, 2009

Related U.S. Application Data
(63) Continuation-in-part of application No. 12/348,947, filed on Jan. 6, 2009.
(60) Provisional application No. 61/020,558, filed on Jan. 11, 2008.
(60) Provisional application No. 61/023,905, filed on Jan. 28, 2008.

(51) Int. Cl. A61K 36/45; A61P 31/04

(52) U.S. Cl. 424/732

(57) ABSTRACT

A method is disclosed that prevents, controls and ameliorates urinary tract infections caused by E. coli by administering a therapeutically effective amount of a human dietary supplement composition comprising a cranberry derivative or proanthocyanidin containing concentrate and D-mannose combined in a form suitable for oral consumption. The cranberry derivative or proanthocyanidin containing concentrate comprises from about one percent (1.0%) by weight to about 95 percent (95.0%) by weight on a dry weight basis. The composition is formulated for delivering a D-mannose unit dosage between about 0.5 to about 5.0 grams per dose and further comprising an analgesic or antispasmodic or combination thereof, and optionally a diuretic, and wherein the composition formed from the cranberry derivative or proanthocyanidin containing concentrate, D-mannose are effective together for binding to E. coli to aid in flushing the E. coli from the urinary tract system while preventing binding of E. coli to cells in the urinary tract system.

### RELATED APPLICATIONS

This application is a continuation-in-part application of U.S. patent application Ser. No. 12/348,947, filed Jan. 6, 2009, which is based upon U.S. provisional patent application Ser. No. 61/020,558 filed Jan. 11, 2008 and U.S. provisional patent application Ser. No. 61/023,905 filed Jan. 28, 2008.

### FIELD OF THE INVENTION

The present invention relates preventing, controlling and ameliorating urinary tract infections (UTI) using cranberry derivative and D-Mannose compositions.

### BACKGROUND OF THE INVENTION

Urinary tract infections (UTIs) are generally defined as the presence (>100,000/mL) of bacteria in the urine. UTIs are commonly caused by Gram-negative bacteria, particularly Escherichia coli (E. coli), and infect primarily women. This infection is enabled by the adherence and colonization of bacteria to urinary tract epithelial cells. Adherence by E. coli is performed by proteinaceous fibers (fimbriae) on the bacteria cell wall, which attach to specific oligosaccharide receptors on uroepithelial cells. Antibiotics are commonly prescribed for treatment, but often promote bacterial resistance. One in four women also encounter recurrence of the infection and are often found to be prone to such infections. Natural substances which could treat and prevent UTIs could be useful for those suffering this condition since antibiotic treatment, in many cases causes, a secondary vaginal yeast infection requiring a subsequent antifungal treatment.

Consumption of cranberries has been found to be somewhat effective in addressing UTI infections. Cranberry products can prevent adhesion of certain bacteria fimbriae to uroepithelial cells in the urinary tract, thereby reducing the ability of the bacteria to create an infection (DiMartino et al., “Reduction of Escherichia Coli Adherence to Uroepithelial Bladder Cells After Consumption of Cranberry Juice: A Double-Blind Randomized Placebo-Controlled Cross-Over Trial,” World Journal of Urology, 2006); (Liu et al., “Role of Cranberry Juice on Molecular-Scale Surface Characteristics and Adhesion Behavior of Escherichia Coli,”, Biotechnology Bioengineering, 2006). Proanthocyanidins (condensed tannins) found in the cranberry juice have been shown to inhibit E. coli adherence (Howell et al., “Inhibition of the Adherence of P-Fimbriated Escherichia Coli to Uroepithelial-Cell Surfaces by Proanthocyanidin Extracts from Cranberries,” Journal of Medicine, 1998). Some E. coli fimbriae bind specifically to D-mannose and their binding unaffected by cranberry based ingredients including proanthocyanidins. D-Mannose is a simple monosaccharide (a simple sugar) with unusual characteristics. D-mannose, unlike sucrose or fructose, is metabolized very slowly in humans, therefore once consumed, D-mannose will enter the blood stream and quickly move to excretion via the kidneys followed by entry into the bladder in urine. D-mannose once in urine will cause the bacterial fimbriae sensitive to D-mannose binding to attach to the D-mannose, rather than epithelial cells. This allows the body to flush the D-mannose bound E. coli bacteria from the body. In addition, D-mannose can reverse epithelial bound E. coli competitively interrupting the initial phases of urinary tract infection. To mitigate existing UTIs and prevent recurrence, regular consumption of cranberry in combination with D-mannose will prevent bacteria from adherence, colonization and ultimately prevent an uncontrollable urinary tract infection. For this strategy to work, consumer compliance is necessary. D-mannose has a natural sweetness, and cranberry juice and its derivatives generally possess acceptable flavors.

Combinations as compositions using cranberry have been presented by others, for example, in GB2396811 (WO 2004/056380), the disclosure which is hereby incorporated by reference in its entirety. As noted in cited reference, described compositions include an extract from a plant that is a member of the Ericaceae, Rosaceae, Pinaceae or Vitaceae family and at least one sugar that is not metabolized or is only partly metabolized by the human or animal body. The sugar is preferably a monosaccharide such as L-arabinose, L-fucose, D-mannose, L-rhamnose, L-xylose, lyxose or galactose. A preferred composition includes an extract of cranberry with D-mannose. These compositions may be used to treat bacterial infection caused by E. coli, particularly urinary tract infections (UTIs). Compositions also include an anthocyanin, anthocyanidin or a proanthocyanidin and at least one sugar that is not metabolized or is only partly metabolized by the human or animal body are also described.

Example of D-mannose compositions are also disclosed in U.S. Patent Publication Nos. 2007/0166292 and 2007/0244069; and U.S. Pat. Nos. 5,525,341 and 6,210,681, the disclosures of which are hereby incorporated by reference in their entirety.

### SUMMARY OF THE INVENTION

Cranberry derivatives and D-mannose are combined in a novel and unobvious concentration and proportion with various additives for preventing, controlling or ameliorating urinary tract infections caused by E. coli by administering a therapeutically effective amount of a human dietary supplement composition as a cranberry derivative or proanthocyanidin containing concentrate and D-mannose combined in a form suitable for oral consumption. The cranberry derivative or proanthocyanidin containing concentrate is formed from about one percent (1.0%) by weight to about 95 percent (95%) by weight on a dry weight basis and formulated for delivering a D-mannose unit dosage between about 0.5 to about 5.0 grams per dose. At least one diuretic additive is also added such that the composition is effective for binding to the E. coli and to aid in flushing the thus bound E. coli from the urinary tract.

In one aspect, the D-mannose is derived from a natural or synthetic source. The cranberry derivative can be derived from whole cranberries, juice, puree, extract, dried powder concentrate, seed extract, or any combination thereof. A carrier can also be administered for example, maltodextrin, starch, cellulose, food-grade silica or flow agents, or one or more acidulants, for example citric acid, malic acid or ascorbic acid. Acidulants are useful in lowering the pH of urine, a method known to reduce the susceptibility to infectious microbes.

In another aspect, the composition is prepared and packaged in a wet or dry form suitable for direct addition to water as a beverage pre-mix concentrate. Other food ingredient components can be added to increase the palatability of the formula, including, for example, natural and/or artificial flavors, nutritive and/or non-nutritive sweeteners, salts, acids or other suitable food ingredients. The composition can be incorporated into a solid or semi-solid food or a beverage. In one aspect, the composition is added to a liquid as a ready-to-drink beverage. The composition in another aspect can be provided as tablets, capsules, powders, emulsions, liquid concentrates, beverages, confectionary candies, including gummy bear confectionaries or chocolate, or encapsulated in liquid gels.

Other biologically active extracts and compounds can be added, including for example, vitamins, minerals, antioxidants, dietary fibers, tocopherols, tocotrienols, phytosterols, polysaccharides, polyphenolics, bioflavonoids or, in a dry formulation with probiotics known to improve vaginal health. The composition can contain a naturally occurring diuretic including but not limited to, for example, saw palmetto, uva ursi, asparagus root, goldenrod, parsley, cleavers, dandelion, hydrangea, or any combination thereof. The composition can also contain a prescription diuretic, including but not limited to, for example, chlorothiazide, furosemide, theobromine, theophylline, oleandrin, or amiloride.

A capsule can contain the cranberry derivatives and D-mannose and other additives in an effective dosage form. The composition can be advantageously densified into a roller compacted powder to increase bulk density and decrease effective dose volume.

The proanthocyanidin containing concentrate ingredient of the invention can be derived from blueberry, grape, French maritime bark extract or any combination thereof either alone or as a juice concentrate bound to naturally occurring fibers, including but not limited to cranberry pomace derived from drying wet press cake from the juicing of fresh cranberries. In another aspect of the invention a probiotic can be added, for example Lactobacillus spp. that competes and has activity against undesirable bacteria, including E coli. Examples of probiotics include but are not limited to strains of the genera Lactobacillus, Bifidobacterium, Bacillus or Lactobacillus. Such probiotics promote good vaginal health by acidification of the mucous membranes thereof which prevent migration of E. coli from the anal area to the urinary tract entrance. Golden seal (Hydrastis canadensis) can be added for increasing IgM production. Stinging nettle (Urtica dioica) can be added as a diuretic. Echinacea (Echinacea spp.) can be added for stimulating anti-inflammatory effects and immunomodulatory and immunostimulant activity and has been found effective for use in the treatment method and composition.

In order to ameliorate pain, an analgesic may also be added, for example, phenazopyridine hydrochloride, aspirin, phenacetin, or any of the NSAID class of analgesics or a narcotic such as codeine, butorphenol and the like or with antispasmotics to reduce the pain and discomfort associated with urinary tract infections.

### DETAILED DESCRIPTION OF THE PREFERRED EMBODIMENTS

The present invention will now be described more fully hereinafter, in which preferred embodiments are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art.

D-Mannose is a simple sugar and related as a (steroisomer) to D-glucose. D-mannose is metabolized very slowly in humans allowing a steady state concentration in blood serum to carry the D-mannose to the kidneys for intact elimination in urine. The urinary tract infection occurs when E. coli enters the urethra and binds to the inner walls of the bladder, often reaching as far as the kidneys where a more dangerous infection can lead to kidney cell damage. The cell walls of the E. coli are covered with tiny finger like projections called fimbriae whose structures are important to cell wall recognition and subsequent infection forming a “glycoprotein”, also referred to as a “lectin”. D-Mannose competitively binds to the E. coli lectin binding recognition sites thus preventing attachment of uroepithelial cells. Bound E. coli can then be simply eliminated via urination. The inventors believe that a non-antibiotic method of eliminating E. coli from the urinary tract may be advantageous when considering antibiotic resistance and secondary yeast infections caused by antibiotic treatments. In addition antibiotics kill not only the unwanted micro-organisms but also kill friendly micro-organisms. For example many females suffer from vaginal yeast infections following antibiotic use because the natural bacteria are killed along with the unwanted infectious bacteria and/or fungi, leaving antibiotic insensitive fungi, such as Candida albicans, to grow and reproduce further complicating the treatment to health. Long-term antibiotic use can lead to major disruptions in normal body microflora, disrupts health and is known to promote bacterial antibiotic resistance.

It has been found that the D-Mannose in combination with cranberry proanthocyanin concentrates can be helpful in these situations. In accordance with a non-limiting aspect of the present invention, the use of a cranberry derivative or proanthocyanidin containing concentrate in combination with D-Mannose and a diuretic and optionally other effective additives in a therapeutically effective amount with proper proportions is a suitable composition for oral consumption and complements the effect of D-Mannose since for example, E. coli populations contain both D-mannose sensitive and D-mannose insensitive binding and recognition sites. For example, the cranberry derivative or proanthocyanidin containing concentrate can be about one percent by weight to about 95 percent by weight on a dry weight basis and the total composition formulated for delivering a D-mannose unit dosage between 0.5 and 5.0 grams per dose. Although natural cranberry juice contains small amounts of D-Mannose the supplemental composition containing the cranberry derivative or proanthocyanidin containing concentrate or multiple cranberry derivative(s) and D-Mannose with a diuretic is effective and advantageous as a dietary supplement composition when each are proportioned in a specific manner.

In one non-limiting aspect, the cranberry derivative(s) are derived from whole cranberries, juice, puree, extract, dried powder concentrate, seed extract, or any combination thereof.

The composition is incorporated with a suitable carrier such as maltodextrin, starch, cellulose, food-grade silica, flow agents, and one or more acidulants such as citric acid, malic acid and ascorbic acid in a non-limiting example. The composition as a formula can be prepared and packaged in a wet or dry form suitable for direct addition to water to form a beverage drink. Other additives to the drink can also be used.

In one non-limiting and preferred aspect, the composition as a formulation delivers a D-mannose unit dosage between about 0.5 and about 5.0 grams per dose. As another non-limiting example, the cranberry derivative or proanthocyanidin containing concentrate typically comprises from about one percent (1.0%) by weight to about 95 percent (95.0%) by weight of the formula on a dry weight basis. This proportion is therapeutically effective and advantageous.

Other components can be added to increase the palatability of the formula, including for example, natural and/or artificial flavors, nutritive and/or non-nutritive sweeteners, salts, acids or other suitable food ingredients. The compositions can be incorporated into food and beverage, for example, as a human dietary supplement composition in a ready to drink beverage.

The compositions can also be in the form of tablets, capsules, powders, emulsions, liquid concentrates, beverages, confectionary candies and liquid gels. Other biologically active extracts and compounds can be added including for example, vitamins, minerals, antioxidants, dietary fiber, tocopherols, tocotrienols, phytosterols, polysaccharides, polyphenolics and bioflavonoids, analgesics or antispasmodics and have been found to add to the therapeutically effectiveness of the treatment method and composition.

The formula as a composition can contain a naturally occurring diuretic such as saw palmetto, uva ursi, asparagus root, goldenrod, parsley, cleavers, dandelion, hydrangea, and the like and extracts of such and in different combinations.

The formula as a composition can also contain a prescription diuretic such as chlorothiazide, furosemide, theobromine, theophylline, oleandrin, amiloride and analgesic or an antispasmotic as previously described.

An analgesic or antispasmodic can be added such as scopolamine, phenazopyridine hydrochloride, aspirin, acetaminophen and any of the class of NSAIDS or a herbal such as scopolia root, angelica root and the like to reduce the pain of E. coli urinary tract infections.

Additionally, the formulation may be compacted in suitable roller compaction device in order to increase the bulk density, thereby reducing the consumption volume needed for an effective dose. It is known that consumer dose compliance decreases with increasing capsule size and number, therefore formulation compaction can increase consumer compliance and resulting effectiveness, particularly in capsule presentations. For example, the composition can have a higher density, resulting in an effective therapeutic dosage using two capsules instead of four capsules when capsules are used in the method of treatment.

The proanthocyanidin containing concentrate can be derived from blueberry, grape, French maritime bark extract and D-mannose or any combination thereof. In another aspect a probiotic can be added, for example, Lactobacillus spp. that produces lactic acid in the gut and therefore is known to acidify the vaginal mucosa. Such acidity has activity against undesirable bacteria, including E. coli. Examples of lactobacillus spp. include acidophilus, rhamnosus, reuteri, casei or sporogenes spp. Other probiotics such as strains of the genera Bifidobacterium or Bacillus may offer benefit. Golden seal (hydrastis Canadensis) can be added for increasing IgM production. Stinging nettle (Urtica dioica) can be added as a diuretic. Echinacea (echinecea spp.) can be added for stimulating anti-inflammatory effects and immunomodulatory and immunostimulant activity.

As noted above, goldenseal acts to increase IgM and similar components include Mahonia (Oregon grape) and Berberis (Barberry). It is believed that these components have the ability to inhibit drug resistance efflux pumps (MDR pumps) of bacteria. Goldenseal contains isoquinoline alkaloids: hydrastine, berberin, berberastine, hydrastinine, tetrahydroberberastine, canadine, and canalidine. Possibly the action of 8-oxotetrahydrothalifenine is operative Berberine and hydrastine are believed to act as quaternary bases.

Proanthocyanidins as identified above can also be found in many plants, for example, apples, pine bark, cinnamon, grape seed, cocoa, grape skin, and red wines of Vitis vinifera. Bilberry, cranberry, black currant, green tea, black tea and other plants also contain these flavonoids. The berries of chokeberry, such as black chokeberry, have high concentrations of proanthocyanidin and can be used.

Proanthocyanidins are known to bind to p-pilitated E. coli which are insensitive to binding by D-mannose. They can act also as vasoactive polyphenols such as in red wine and reduce the risk of coronary heart disease. They are believed to suppress production of a protein endothelin-1 that constricts blood vessels. Proanthocyanidins are believed to have antioxidant activity and stabalize collagen maintenance of elastin—two critical proteins in connective tissue that support organs, joints, blood vessels, and muscle. Proanthocyanidins are also believed to reduce histamine production and beneficial in treating allergies while also improving circulation by strengthening capillary walls. They are also believed to inhibit enzymes that break down collagen and help collagen repair and act as a sunscreen. Proanthocyanidins can cross the blood-brain barrier to fight free radicals in the vessels of the brain and increase mental acuity.

As to the diuretic action of stinging nettle, some studies show that a sex hormone binding globulin (SHBG), aromatase, epidermal growth factor and prostate steroid membrane receptors are involved in the anti-prostatic effect. It is not clear that 5α-reductase or androgen receptors are used. Some anti-inflammatory activity may be affected by extract and a polysaccharide fraction. It is believed also to contain different acids, for example, silicic, threonic, and formic acids and contain some amines such acetylcholine, choline, serotonin and histamine as well as flavonoids. A polysaccharide fraction could also aid in an anti-inflammatory effect and polypeptide fraction. Hops contain alpha- and beta-acids, where the alpha-acids occur as humulone, cohumuline and adhumulone, and essential oils and prenylflavonoids.

Hops can have a sedative effect and also act as a potent estrogen receptor agonist in estrogen-responsive cells and aid in treating menstrual symptoms. Bitter acids in hops have an antibacterial and antifungal activity. Marshmallow root is believed to have bactericidal and anti-inflammatory properties. Myrrh is typically found as an oleo-gum and includes a volatile oil of sesquiterpenes, sterols, and steroids and can be used for antiparasitic effects and infections for females and males. It is believed that honey bee pollen contains some Apalbumin1 (Apa1) as a royal jelly (RJ) and honey glycoprotein having various biological properties. It could possibly stimulate macrophages to release tumor necrosis factor alpha (TNFalpha) and possibly has immuno-stimulatory effects. Plantain leaf is useful in GI therapy and treats hyperlipidemia through various actions. It includes various acids with various flavonoids.

Oregon Grape has various alkaloids, including berberine, berbamine, canadine, and hydrastine and can treat diarrhea in patients infected with E. coli, such as by slowing the transit time in the intestine and possibly inhibiting the ability of bacteria to attach to human cells, which helps prevent infections in the intestines and urinary tract. Echinacea has antibacterial properties possibly selectively modulates the catalytic activity of CYP3A at hepatic and intestinal sites.

Many modifications and other embodiments of the invention will come to the mind of one skilled in the art having the benefit of the teachings presented in the foregoing description. Therefore, it is understood that the invention is not to be limited to the specific embodiments disclosed, and that modifications and embodiments are intended to be included within the scope of the appended claims.

1. A method of preventing, controlling and ameliorating urinary tract infections caused by E. coli by administering a therapeutically effective amount of a human dietary supplement composition comprising a cranberry derivative or proanthocyanidin containing concentrate and D-mannose combined in a form suitable for oral consumption, wherein the cranberry derivative or proanthocyanidin containing concentrate comprises from about one percent (1.0%) by weight to about 95 percent (95.0%) by weight on a dry weight basis and said composition is formulated for delivering a D-mannose unit dosage between about 0.5 to about 5.0 grams per dose and further comprising an analgesic or antispasmodic or combination thereof and wherein the composition formed from the cranberry derivative or proanthocyanidin containing concentrate and D-mannose in the presence of the analgesic or antispasmodic or combination thereof are effective together for binding to E. coli and eliminating E. coli from the urinary tract system.

2. The method of claim 1, wherein the D-mannose is derived from a natural or synthetic source.

3. The method of claim 1, wherein the cranberry derivative is derived from whole cranberries, juice, puree, extract, dried powder concentrate, seed extract, or any combination thereof.

4. The method of claim 1, and further comprising administering a carrier comprising maltodextrin, starch, cellulose, food-grade silica or flow agents, one or more acidulants comprising citric acid, malic acid or ascorbic acid or any combination thereof.

5. The method of claim 1, further comprising preparing and packaging the composition in a wet or dry form suitable for direct addition to water as a beverage pre-mix concentrate.

6. The method of claim 1, and further comprising adding food components to increase palatability comprising natural or artificial flavors, nutritive or non-nutritive sweeteners, salts, or acids or any combination thereof.

7. The method of claim 1, and further comprising incorporating the composition in a food or beverage.

8. The method of claim 7, and further comprising incorporating the composition into a ready to drink beverage.

9. The method of claim 1, and further comprising forming tablets, capsules, powders, emulsions, liquid concentrates, beverages, confectionary candies or liquid gels to which the composition is contained.

10. The method of claim 1, and further comprising adding biologically active extracts and compounds, comprising vitamins, minerals, antioxidants, dietary fibers, tocopherols, tocotrienols, phytosterols, polysaccharides, polyphenolics or bioflavonoids, analgesics, antispasmodics or any combination thereof.

11. The method of claim 24, wherein the diuretic comprises a naturally occurring diuretic.

12. The method according to claim 11, wherein said naturally occurring diuretic comprises stinging nettle, saw palmetto, uva ursi, asparagus root, goldenrod, parsley, cleavers, dandelion, hydrangea, or any combination thereof.

13. The method of claim 24, wherein the diuretic comprises a prescription diuretic.

14. The method according to claim 13, wherein the prescription diuretic comprises chlorothiazide, furosemide, theobromine, theophylline, oleandrin or amiloride.

15. The method of claim 1, and further comprising adding an analgesic or antispasmodic comprising scopolamine, phenazopyridine hydrochloride, aspirin, or acetaminophen or NSAID or any combination thereof or a herbal comprising scopolia root or angelica root or any combination thereof for reducing the pain of E. coli urinary tract infections.

16. The method of claim 1, and further comprising forming a capsule containing the cranberry derivative or proanthocyanidin concentrate and D-mannose in an effective dosage form.

17. The method of claim 1, and further comprising roller compacting the cranberry derivative or proanthocyanidin containing concentrate and D-mannose and optionally a binder additive to increase bulk density and decrease effective dose volume.

18. The method according to claim 1, wherein the proanthocyanidin containing concentrate is derived from a member of Vaccinium genera, blueberry, grape, or French maritime bark extract or any combination thereof.

19. The method according to claim 1, and further comprising adding a probiotic comprising Lactobacillus spp. that improves the acidity of urine and vaginal mucosa and therefore prevents further migration of E. coli to the site of infection.

20. The method according to claim 18, wherein the step of adding Lactobacillus spp. comprises adding acidophilus, rhamnosus, reuteri, casei or sporogenes spp. or any combination thereof.

21. The method according to claim 1, and further comprising adding golden seal (Hydrastis canadensis) for increasing IgM production.

22. The method according to claim 1, and further comprising adding an herbal medicine for treating urinary infections or inflammation comprising asparagus root, birch leaf, uva ursi, stinging nettle or any combination thereof.

23. The method according to claim 1, and further comprising adding Echinacea (Echinacea spp.) for stimulating anti-inflammatory effects and immunomodulatory and immunostimulant activity.

24. The method according to claim 1, and further comprising adding a diuretic.
